(19) United States
(12) Patent Application Publication
(10) Pub. No.: US 2021/0324442 A1
(43) Pub. Date: Oct. 21, 2021

(54) In vitro Transcription-Translation Metabolic Networks to Measure Quantity or Activity of Enzymes

(71) Applicant: Brigham Young University, Provo, UT (US)

(72) Inventors: Bradley C. BUNDY, Provo, UT (US); J. Porter HUNT, Provo, UT (US)

(21) Appl. No.: 17/142,932

(22) Filed: Jan. 6, 2021

Related U.S. Application Data
(60) Provisional application No. 63/010,247, filed on Apr. 15, 2020.

(51) Int. Cl. C12Q 1/48

(52) U.S. Cl. CPC C12Q 1/485; G01N 2333/9124

(57) ABSTRACT

Disclosed herein are biosensors including transcription, translation, and coupled transcription/translation systems. The biosensors may be made from cell lysates, purified enzymes, or a combination thereof. The biosensors may include an inhibitor, and may include a reporter. The biosensor may be supplied in a kit for testing for a disease or condition. Methods of using the biosensor are also disclosed.

### CROSS-REFERENCE OF RELATED APPLICATIONS

The present application claims the benefit of and priority to U.S. Provisional Application No. 63/010,247, filed Apr. 15, 2020, the contents of which are incorporated herein by reference in their entirity.

### FEDERALLY SPONSORED RESEARCH STATEMENT

This invention was made with government support under Grant Number CBET-1254148 awarded by the National Science Foundation. The government has certain rights in the invention.

### BACKGROUND

The present disclosure relates to systems, kits, and methods useful for analyzing biological specimens, particularly for sensing metabolites in samples for research or diagnostic purposes.

The amino acid L-glutamine (Gln) serves physiological functions in metabolism, intestinal health, immune function, and cell signaling. Altered serum Gln concentration has been associated with a variety of disorders and diseases, including severe infection, anorexia nervosa, chronic kidney disease, and diabetes. Additionally, Gln metabolism in cancer is attracting increasing attention. Studies suggest that degradation of Gln by asparaginase may be a component of the latter's anti-leukemic efficacy. Recent work on cancer Gln metabolism has led to clinical trials target Gln in treatment for non-small cell lung cancer, renal cancer, colorectal cancer, melanoma, myelodysplastic syndrome, and metastatic solid tumors and suggests that modulating Gln metabolism could be a hallmark of future cancer treatment. Thus, quantifying serum Gln levels is a potential tool for a number of disease diagnoses and treatments.

Despite this clinical potential, measuring serum Gln levels is challenging. Current methods rely on high-performance liquid chromatography or magnetic resonance spectroscopy, which require extensive preparation and expensive overhead equipment, precluding routine clinical use.

### BRIEF SUMMARY

In one aspect, the present disclosure provides in vitro system for measuring a biological activity in a sample. The in vitro system may include at least one of a transcription system and a translation system; an inhibitor of the transcription system or the translation system; and a reporter of the biological activity. The reporter is inactive when the sample is absent, and the reporter provides a signal proportionate to rescue of the biological activity by the sample.

In one aspect, the present disclosure provides an in vitro system for measuring a biological activity in a sample. The in vitro system may include a coupled transcription-translation system; an inhibitor of the coupled transcription-translation system; and a reporter of the biological activity. The reporter is inactive when the sample is absent, and wherein the reporter provides a signal proportionate to rescue of the biological activity by the sample.

In another aspect, the present disclosure provides a method of using an in vitro system to detect a metabolite or a metabolic activity. The method may include combining the sample with the in vitro system, and quantifying the signal produced after a predetermined time.

### BRIEF DESCRIPTION OF THE DRAWINGS

Reference is made to the following drawings, wherein:

FIG. 1 is (A) a schematic view of a cell free protein synthesis (CFPS) process as described herein and (B) a graph quantifying a green fluorescent protein (GFP) signal produced by one such synthesis;

FIG. 2 is a graph showing CFPS expression of GFP in relative fluorescence units (RFU) with 0 millimolar (mM) and 2 mM initial L-asparagine, with error bars representing one standard deviation for n=2 CFPS reactions;

FIG. 3 includes (A) schematics of CFPS processes in accordance with one aspect of the present disclosure and (B) a graphical representation of the results of a glutamine sensor assay conducted with such a CFPS system;

FIG. 4 is an image of a denaturing electrophoretic gel running the product of ¹⁴C-labeled crisantaspase-producing CFPS reactions with an autoradiogram overlay;

FIG. 5 is a graph representing the response of CFPS reactions producing GFP initiated with 2 mM L-aspartate (ASP) and the indicated E. coli L-asparaginase activity;

FIG. 6 is a graph representing the results of a CFPS asparaginase assay with increasing concentrations of purified asparaginase added;

FIG. 7 is a graph representing results of CFPS Gln sensor assay in the presence of human serum; and

FIG. 8 is a graph representing the results of CFPS reactions producing GFP in the presence of ASP and asparaginase.

### DETAILED DESCRIPTION

As used herein, the term “about,” means “approximately but not necessarily equal to,” and when used in the context of a numerical value or range set forth means a variation of ±15%, or less, of the numerical value. For example, a value differing by ±15%, ±12%, ±10%, or ±5%, among others, would satisfy the definition of “about.”

As used herein, the term “sample” refers to biological material testable in a system, kit, or mixture described herein.

As used herein, a “patient” is an animal, particularly a mammal, and most particularly a human, from which a sample is or can be collected.

Disclosed herein is a rapid, portable, and economic alternative to existing measurement methods for the activity or quantity of enzymes, enzyme inhibitors, or transcription- and translation-dependent biomolecules. In one example, a rapid method for quantifying serum glutamine levels in patients with anorexia nervosa is disclosed. In another example, a rapid method for quantifying serum asparaginase levels in patients undergoing leukemia chemotherapy is disclosed.

In one aspect, dehydrated, bacterial-based, cell-free protein synthesis sensor systems are proposed. Tests run with such sensor systems can be rapid (returning results in about an hour), are very economical, require only very small sample sizes (μ1 range), and are portable. This stands in contrast to conventional methods that are more expensive owing to the inclusion of liquid chromatography and/or nuclear magnetic resonance steps. These workflows are confined to a laboratory, and/or require many hours and/or large sample volumes to execute.

Bioavailable glutamine (Gln) is critical for metabolism, intestinal health, immune function, and cell signaling. Routine measurement of serum Gln concentrations could facilitate improved diagnosis and treatment of severe infections, anorexia nervosa, chronic kidney disease, diabetes, and cancer. Current methods for quantifying tissue Gln concentrations rely mainly on HPLC, which requires extensive sample preparation and expensive equipment. Consequently, patient Gln levels may be clinically underutilized. Cell-free protein synthesis (CFPS) is an emerging sensing platform with promising clinical applications. In this work, in vitro amino acid metabolism is engineered with methionine sulfoximine to inhibit glutamine synthetase and create a CFPS Gln sensor. The sensor features a strong signal-to-noise ratio and a detection range ideally suited to physiological Gln concentrations. Furthermore, it quantifies Gln concentration in the presence of human serum. This work demonstrates that CFPS reactions which harness the metabolic power of E. coli lysate may be engineered to detect clinically relevant analytes in human samples. Such approaches could lead to transformative point-of-care diagnostics and improved treatment regimens for a variety of diseases including cancer, diabetes, anorexia nervosa, chronic kidney disease, and severe infections.

The present disclosure provides an in vitro transcription, translation, or coupled transcription/translation system. Such a system may be assembled from cell extract, from purified enzymes and other components (such as transfer RNA (tRNA), amino acids, and the like), or from a combination of cell extract and purified enzymes and other components.

A “transcription system” is a collection of biomolecules that is competent to generate an RNA transcript from a DNA molecule, including RNA polymerases, nucleotides and their derivatives, and so forth. A “translation system” is a collection of biomolecules competent to generate a polypeptide from an RNA transcript, including ribosomes, amino acids, transfer RNAs (tRNAs), and so forth.

The system may be a cell-free protein synthesis (CFPS) system. To the system may be added an enzyme or an enzyme inhibitor in order to induce a metabolic deficiency in the transcription, translation, or coupled transcription/translation reaction chain. The rate or extent of transcription or translation is a measure or sensor of enzyme or inhibitor quantity or activity. Transcription and/or translation can be correlated to the level of synthesis or activity of an included reporter, which can generate a signal proportionate to, or inversely proportionate to, enzyme or inhibitor quantity or activity. The system may be a dehydrated mixture of any of the preceding.

In another aspect, the transcription, translation, or coupled transcription/translation system may be exposed to a sample, such as a biological sample obtained from a patient suspected of having a disease, condition, or disorder, or from a patient undergoing treatment for a known disease, condition, or disorder. In this aspect, the rate or extent of transcription or translation is a measure of the quantity or activity of the rescuing factor in the sample.

In one aspect, an in vitro coupled transcription-translation reaction deficient in glutamine may be assembled with bacterial cell extract, specifically Escherichia coli (E. coli) cell extract. An inhibitor of the native E. coli glutamine synthetase is also added to the reaction. A sample with an unknown quantity of glutamine is added to the reaction. The reporter is a green fluorescent protein (GFP) gene, provided as linear or circular DNA, or as messenger RNA (mRNA). The rate or extent of GFP synthesis as indicated by reaction fluroescence is a measure of the quantity of gluamine in the sample.

In a different aspect, an in vitro coupled transcription-translation reaction is assembled with E. coli cell extract. A sample containing the cancer therapeutic asparaginase, which degrades asparagine, is also added to the reaction. The rate or extent of reporter signal (for example, GFP synthesis as indicated by reaction fluorescence) is a measure of the activity of asparaginase in the sample.

In a further aspect, an in vitro coupled transcription-translation reaction is assembled with E. coli cell extract. Asparaginase is also added to the reaction to counter the activity of the native E. coli asparagine synthetase and create an asparagine deficiency in the metabolic network. A sample with an unknown quantity of asparagine is also added to the reaction. The rate or extent of reporter signal, such as GFP synthesis as indicated by reaction fluorescence, is a measure of the quantity of asparagine in the sample.

Commercially available enzyme assays for metabolites, particularly for L-glutamine (Gln), remain expensive and are subject to the confounding effects of ammonia, an elevated serum constituent during therapeutic Gln depletion. As a consequence, Gln quantification has not been utilized for diagnosing and treating these diseases, and alternative technologies could be transformational.

Cell-free protein synthesis (CFPS) is an emerging sensing platform. The open transcription-translation reaction environment facilitates exquisite system optimization and control, and CFPS biosensors have been engineered to operate on the transcription, translation, and protein-folding levels. Sensing platforms formulated from E. coli lysates leverage the robustness of bacterial gene expression, can be lyophilized to create portable indicators, and have demonstrated utility in a variety of environmental and biological sample matrices. Compared with their microbial counterparts, CFPS biosensors eliminate sample transport limitations and are small, compact, and robust, and readable on common laboratory equipment. They also eliminate the risk of releasing genetically modified microbes and enable more rapid readouts and detection in matrices that are toxic to microbes.

In some aspects, the biosensor may be made from a cell lysate. In some aspects, the lysate may be derived from eukaryotic cells. In other aspects, the lysate may be derived from prokaryotic cells, such as bacterial cells. The bacterial cells can be from any one or more species of bacteria, including Escherichia coli. In some aspects, the biosensor may include purified enzymes or other components, from the same organism as the lysate, or from a different organism, or artificial enzymes.

Recent work demonstrated complementary CFPS reactions for quantifying amino acids and enzymatic activities. However, this approach was unable to quantify Gln concentrations due to the residual activity of native metabolic pathways. In this work, we selected residual E. coli amino acid metabolism using an irreversible Gln synthetase inhibitor to create a CFPS sensor for Gln. We also demonstrated the utility of this approach by validating the Gln assay in human serum in one hour. Point-of-care quantification of serum Gln enabled by this CFPS approach could be exceedingly useful for diagnosing and treating individuals suffering from conditions including or not limited to infection (including severe infection), anorexia nervosa, chronic kidney disease, diabetes, and cancer.

In some aspects, the biosensor system may be used to study the levels of a molecule, such as glutamine, in a blood sample. The sample may be whole blood, or the sample may be serum, such as human serum. Surprisingly, the biosensor as disclosed herein is robust and sensitive when the sample is serum, which has a complex biochemical environment. In some aspects, the system can be used to detect and/or quantify asparaginase activity in human blood and/or human serum.

In some aspects, an RNase inhibitor can be used in conjunction with the biosensor. The RNase inhibitor can be provided with the biosensor system in the kit, either separately from the biosensor mixture itself, or pre-mixed with the system; or it can be added to the sample after collecting; or both. The sample type may be human blood or human serum when the RNase inhibitor is employed.

Samples that can be analyzed by a biosensor or method according to the present disclosure include a wide range of biological substances. Samples can be derived from humans, from non-human animals, or from other organisms, including plants, fungi, single-celled eukaryotes, prokaryotes, and archaea. The biosensor can also analyze purified molecules, including libraries of the same. These molecules include small molecules, metabolites, peptides, nucleic acids, and others.

A biological sample to be analyzed can include body fluids, including blood or any of its components (plasma, serum, and so forth), mucus, sweat, tears, urine, feces, saliva, sputum, semen, gastric fluids, pericardial or peritoneal fluids or washes, throat swabs, pleural washes, ear wax, hair, epithelial cells including skin cells, nails, mucous membranes, amniotic fluid, vaginal secretions, spinal fluid, or any other secretions from the body.

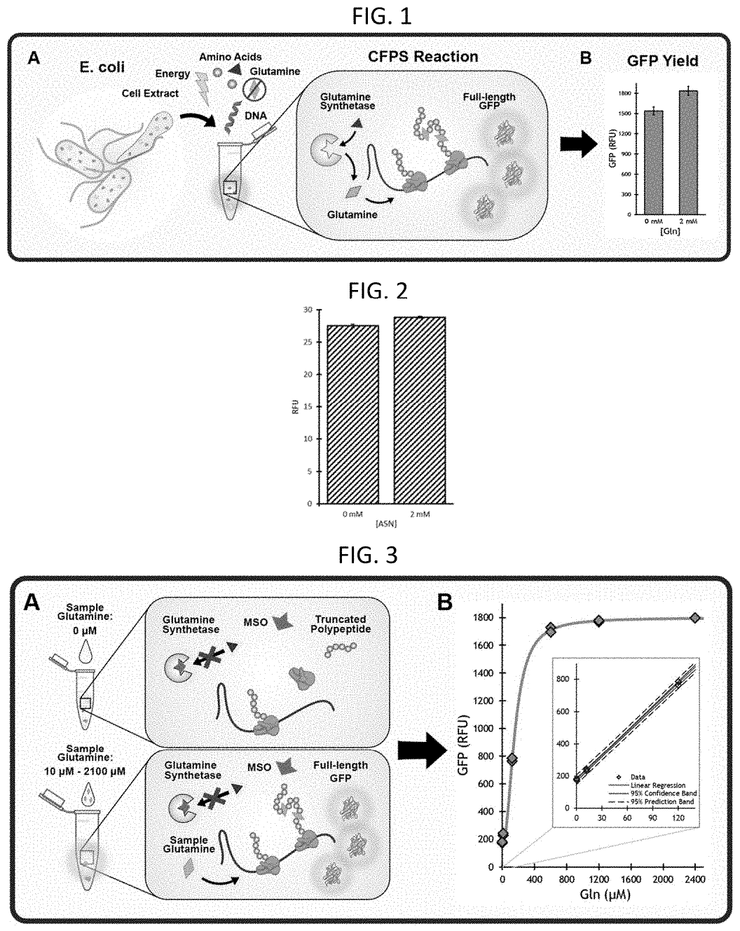

Described herein is a rapid E. coli-based cell-free protein synthesis (CFPS) Gln sensor which could be engineered to deliver point-of-care quantification. First, we initiated GFP-producing CFPS reactions omitting Gln. In principle, the translation machinery of these reactions polymerizes amino acids until the 69^{th} codon of GFP's transcript which specifies Gln. In the absence of tRNA aminoacylated with Gln, the ribosome stalls and a truncated polypeptide is released. In contrast to this expectation, we observed that CFPS reactions omitting Gln achieve similar yields to those with all 20 proteinogenic amino acids (see FIG. 1). This suggests that glutamine is readily synthesized by residual E. coli amino acid metabolism in the CFPS reaction, possibly by Gln synthetase (GLNS).

FIG. 1 illustrates CFPS of GFP and how this may be maintained in a system of the present disclosure when L-glutamine (Gln) is omitted. (A) is a schematic illustration of CFPS, and hypothesized mechanisms of Gln synthesis by native glutamine synthetase (GLNS). Even when Gln is omitted from the system, native GLNS synthesizes it, rescuing protein synthesis. In (B), a graph is displayed, showing GFP fluorescent signal produced by CFPS reactions initiated with or without 2 mM Gln. Data was obtained after 1 hour (hr) reaction and error bars represent one standard deviation for n=2 CFPS reactions.

The abundance of glutamate (>200 mM), combined with residual GLNS activity in CFPS, might produce Gln needed for translation during CFPS. Methionine sulfoximine (MSO) has been identified as a potent irreversible inhibitor of GLNS and we further hypothesized that adding MSO to CFPS could enable the desired Gln detection. We first verified that MSO has little effect on CFPS expression yield at concentrations up to 80 mM in the presence of 2 mM Gln. As seen in FIG. 2, CFPS expression of GFP was measured in relative fluorescence units (RFU) with 0 millimolar (mM, left bar) and 2 mM (right bar) initial L-asparagine, with error bars representing one standard deviation for n=2 CFPS reactions.

Next, we demonstrated that adding MSO to CFPS reactions—where Gln is initially omitted—results in GFP production as a function of the Gln concentration (FIG. 3). This response exhibits a differentiable range below ˜300 μM Gln. The Hill equation fits the Gln sensor data well. As illustrated in FIG. 3, GFP production level by CFPS is dependent on Gln levels when the GLNS-inhibitor MSO is included.

In (A), a schematic illustration of the process of protein synthesis when methionine sulfoximine (MSO), a potent inhibitor of GLNS, is included. In the top panel, when Gln is not added to CFPS and MSO inhibits GLNS, Gln is unavailable for aminoacylation and polypeptide linkage by the ribosome which prevents the synthesis of full-length GFP at high yields. No appreciable fluorescent readout, therefore, is seen. On the bottom, if a sample which contains Gln is added to the MSO-containing CFPS, Gln is more available to the ribosome resulting in higher yields of full-length GFP, and, therefore, a reporter signal can be readily observed.

In FIG. 3, portion (B), results of a CFPS Gln sensor assay are displayed as a bar graph. Reactions omitted Gln as a reagent but included 70 mM MSO. Aqueous samples of known Gln concentrations were added to the CFPS reaction. On the graph, the x-axis represents the final concentration of Gln in the CFPS reaction (ranging from 0 to 2400 μM). Each data point represents a CFPS reaction after 1 hr with n=2 at each Gln concentration. The solid green line represents least-squares regression of the data to the Hill equation, resulting in an EC₅₀ value of 154 μM and a Hill coefficient of 2.1. The inset plots linear regression of the data. The solid blue line represents the fit, solid red bands represent the 95% confidence of fit, and dashed black bands represent the 95% confidence range for a single-point prediction.

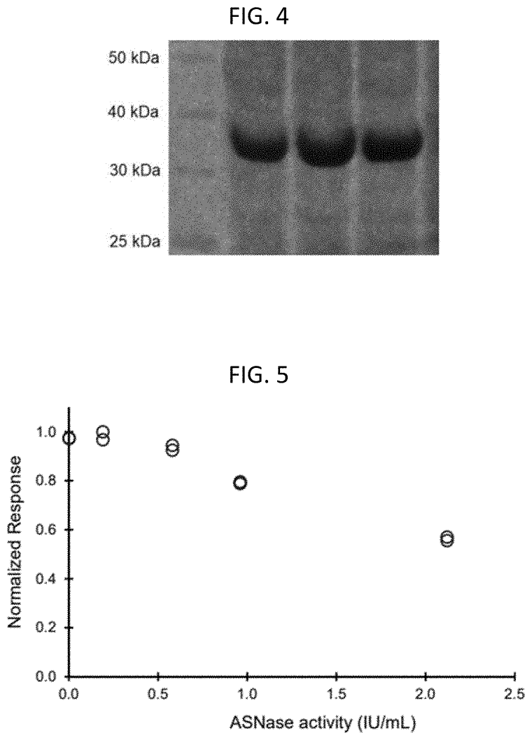

Notably, and as can be seen in FIG. 4, the signal-to-noise ratio of the assay increased linearly to ˜10 with increasing the MSO concentration up to 70 μM . FIG. 4 is an image of a gel of 14C-labeled crisantaspase-producing CFPS reactions, with an autoradiogram overlay. From left to right, lane 1 is protein ladder; lane 2 is 2 mM ASP CFPS; lane 3 is 17 mM ASP CFPS, and lane 4 is 17+ mM ASP CFPS. All lanes were loaded with 4 μl CFPS. For reference, the molecular weight of a crisantaspase monomer is 35.1 kilodaltons (kDa).

The signal-to-noise ratio was also enhanced by increasing overall protein expression yields. The graph of FIG. 5 shows that the response of CFPS reactions producing GFP initiated with 2 mM L-aspartate (ASP) and the indicated E. coli L-asparaginase activity. This reaction format constrasts with those which obtain higher sensitivity to ASNase activity because they are initiated without ASP. GFP yield in the presence of the indicated ASNase activity is normalized to GFP yield in the absence of ASNase activity. Reactions were performed in duplicate and each reaction is represented by a data point.

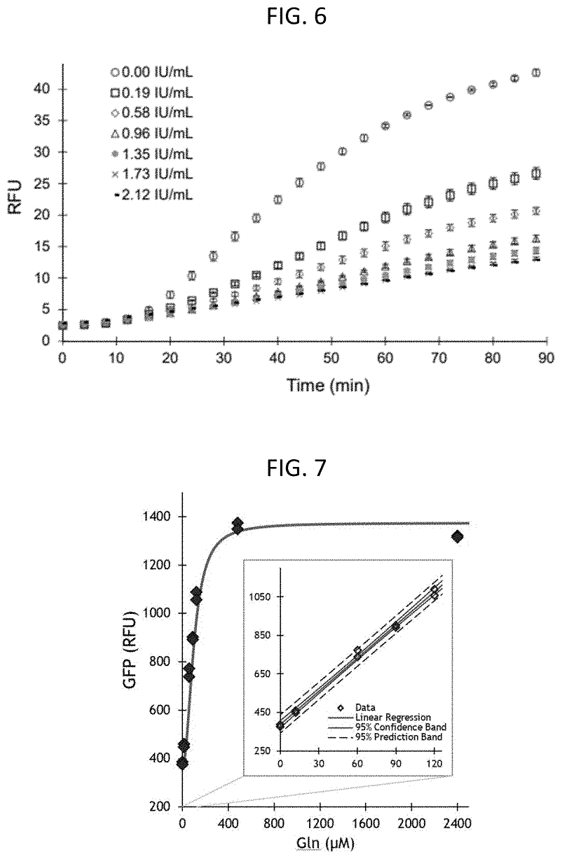

The effect of dialyzing cell extract on CFPS Gln dependence was investigated under the hypothesis that dialysis removes residual free Gln from the extract and might increase the Gln sensitivity of the assay. Dialysis did not improve the limit of detection, however, further supporting the Gln synthesis hypothesis. The results of a CFPS asparaginase assay with increasing concentrations of commercial E. coli asparaginase are presented in FIG. 6. In this trial, the asparaginase was suspended in 290 mOsM HEPES/PEG-400 buffer. The final HEPES/PEG-400 buffer concentration in all CFPS reactions is 47 volume percent. Error bars represent one standard deviation for n=2 CFPS reactions.

The detectable range of this Gln assay is well suited to quantifying physiological Gln concentrations, which may range between 200 and 1400 μM. We tested the performance of this assay in the presence of 14% (v/v) human serum and observed a Gln response nearly identical to reactions initiated with aqueous samples (FIG. 7).

The results of CFPS Gln sensor assay in the presence of human serum are shown in FIG. 7. Reactions omitted Gln as a reagent but contained 70 mM MSO, RNAse inhibitor, and 14% (v/v) human serum with increasing Gln concentrations. The x-axis represents the corresponding Gln concentration in the CFPS reaction. Each data point represents a CFPS reaction after 1 hr with n=2 at each CFPS Gln concentration. The solid blue line represents least-squares regression of the data to the Hill equation, resulting in an EC₅₀ value of 100 μM and a Hill coefficient of 2.1. The inset shows linear regression of the data. Solid blue line represents the fit, solid red bands represent the 95% confidence of fit, and dashed black bands represent the 95% confidence range for a single-point prediction.

This work demonstrates the potential of a human serum Gln assay which requires only microliter quantities of blood and can be executed in an hour. The presented sensor's detectable range corresponds to physiological Gln levels which fall between 200 and 1400 μM. As shown in FIG. 3, the GFP response in CFPS reactions with human serum is linear from 0 to 120 μM Gln and is estimated to be differentiable up to ˜300 (FIG. 7). Only 14% of the CFPS volume is the serum sample, however, such that the detection range for sample Gln is estimated to be up to ˜2100 μM Gln. Additionally, our previous work suggests the amount of serum sample added to the CFPS sensor may be adjusted to any concentration up to 44% (v/v) which would further expand the assay's Gln detection range.

Clinical applications for this sensor, and similar sensors, include making determinations and protocols in cancer treatments which target Gln metabolism. For example, treatment of Acute Lymphoblastic Leukemia (ALL) involves depleting circulating asparagine and Gln with bacterial L-asparaginase, but subsequent patient Gln levels are not routinely monitored nor used to inform individual treatment regimens. In addition, there are currently six clinical trials incorporating the use of a glutaminase inhibitor as a treatment for patients with non-small cell lung cancer, renal cancer, colorectal cancer, melanoma, myelodysplastic syndrome, and metastatic solid tumors. This assay may enhance careful monitoring of Gln during treatment to provide information regarding dosage and administration.

Furthermore, shifts in serum Gln levels may serve as a diagnostic marker for other maladies. For example, the onset of sepsis and critical illness exhibits decreased plasma Gln levels, possibly because Gln is a main fuel source for the immune system. In contrast, chronic kidney disease, anorexia nervosa, and higher incidence of type 2 diabetes are associated with increased serum Gln levels. The CFPS-based assay developed in this work has the potential to mark the onset of such diseases and assist in rehabilitating patients.

In addition to creating a compelling Gln assay, this work also suggests CFPS may be engineered to rapidly screen inhibitors of enzymes. For example, FIG. 4 indirectly reports GLNS inhibition efficiency at varying MSO concentrations.

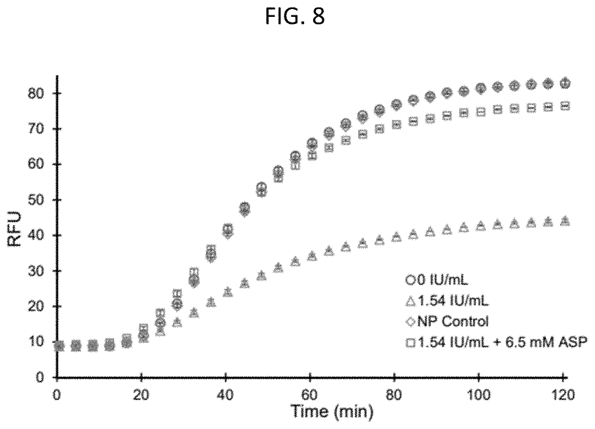

The metabolic engineering approach presented in this work may also be useful for modulating protein expression yield or other desired products from cell-free reactions. Indeed, many enzymes which likely retain activity in the lysate-based CFPS reactions consume precious ATP superfluously. For example, GLNS, which is inhibited in this work, consumes ATP in the process of synthesizing Gln. Notable early work demonstrated precedent when oxalate, an inhibitor of phosphoenolpyruvate synthetase, was shown to increase CFPS expression yield due to enhanced preservation of ATP. Indeed, during the process of completing the work described herein, we learned that phosphoenolpyruvate carboxylase circumvents the production of ATP from PEP, and that its inhibition might improve CFPS performance. Accordingly, we identified Asp as an inhibitor of this enzyme and observed that CFPS initiated with 12 mM Asp compared with the conventional 2 mM Asp increased CFPS yield by 38% over a one-hour reaction (FIG. 8).

In FIG. 8, CFPS reactions producing GFP in the presence of E. coli ASNase and ASP. Reported conditions include 0 IU/mL ASNase (no ASNase added), 1.54 IU/mL ASNase, and 1.54 IU/mL ASNase+6.5 mM ASP. Comparing 1.54 IU/mL and 1.54 IU/mL+6.5 mM ASP curves confirms the inhibitory effect of ASP supplementation on ASNase activity. No plasmid (NP) control tests for the impact of CFPS machinery and possible reaction products if added to a CFPS reaction producing GFP. As such, for the NP control 5 μL of a cellfree reaction product (where no plasmid was added such that no protein expression was templated) was added to a second CFPS reaction producing GFP. This verifies no underlying inhibitory effect if CFPS product is added to the CFPS-based ASNase activity assay. Error bars represent one standard deviation for n=2 CFPS reactions.

The results reported herein demonstrate that using small-molecule inhibitors and amino acid composition to engineer CFPS metabolism is a rich platform for CFPS performance optimization, in addition to sensing and drug discovery.

A feature of the E. coli-based CFPS diagnostic platform demonstrated herein is the economy with which it can be deployed. Each sensor uses only 0.03 mL of CFPS and thus only minor reagent costs. Compared with most current medical diagnostics, this represents a significant cost reduction. With the cost of healthcare increasing, new technologies which enhance the precision and efficiency of medical care at low cost are in high demand. Such technologies may also increase availability to healthcare in underserved communities and the developing world.

Importantly, the human serum sensor reported herein requires only 0.009 mL of serum, which is equivalent to the amount of blood required by commercial, portable glucose meters used by diabetics several times a day. While this initial work reports the use of human serum, we previously reported CFPS-based sensors worked well in whole human blood at 20% (v/v) concentrations. Based on these reports, it is likely that 14% (v/v) whole blood (the volume % of serum used in this work) would perform similarly and eliminate the centrifugation step needed to separate serum.

Finally, we have previously reported lyophilization of CFPS reagents for long-term shelf-stable storage and reported utility for sensing applications, in Salehi et al., “Cell-Free Protein Synthesis Approach to Biosensing hTRb-Specific Endocrine Disruptors,” Anal. Chem. 2017, 89(6), 3395-3401, the entire contents of which are incorporated by reference. Together, these advances suggest glucose-meter-like sensing capabilities for blood Gln concentrations are possible in the near future. Such meters could use blood tests similar to existing technology, taking one drop or so of blood and exposing to the CFPS, and introducing it to a meter, which would then conduct the assay and report the results either on a digital display, or sync with an app on a smartphone or a cloud-based server to provide near-real time monitoring for patients. Any system disclosed herein can be integrated with a computer program of this type.

The biosensor described herein may be provided as part of a kit. The biosensor may be provided as individually-packaged aliquots or doses, and may be provided in liquid format or as a lyophilized or dry mixture. The kit may include other components to assist with analysis, such as sample collection devices including but not limited to swabs, needles, tubes; analytical tools including but not limited to test strips and reagents for the reporter (such as luciferase for a luminescent reporter); and analytical equipment, such as a portable meter with a digital readout. The kit may include printed instructions for use.

Although the present disclosure has mainly discussed GFP as a reporter protein, and fluorescence as a signal for readout, other reporters are contemplated for assays in accordance with the present disclosure. For example, the reporter may be a luciferase gene or a gene of a luciferase variant, with luminescence being the signal generated. In another example, the reporter may give rise to a colorimetric signal, readable in the ultraviolet, infrared, or visible spectra.

In some aspects, the biosensor operates to detect the presence or absence of a molecule, or the activity of an enzyme, in part because transcription or translation in the biosensor is initially impeded, and no signal from the reporter is generated. In general, the impedance of the biosensor system is due to (a) lack of a needed reactant (such as an amino acid, a nucleoside or nucleotide phosphate, or other molecule), (b) lack of a functional enzyme (which may be because the enzyme is absent, or catalytically inactive), (c) presence of an inhibitor (a small molecule, a protein, a chelating agent, or so forth), or (d) a combination of any of these. Thus, the biosensor is intrinsically “off.” When the sample is added, the response of the biosensor may be proportionate to the degree to which the impedance is overcome; that is, how much of the missing reactant, or active enzyme, is supplied by the sample, either to provide it where it was missing, or to overcome the addition of the inbitor, resulting in signal from the reported being turned “on.” This is termed “rescue” of the reaction.

Other diseases, conditions, and disorders may be monitored by systems and methods as described herein.

Although particular attention has been paid to conditions wherein the measurement of glutamine and/or asparagine levels is relevant, other conditions can also be monitored, diagnosed, and otherwise analyzed by systems and methods as described herein, including but not limited to disorders of carbohydrate metabolism such as glycogen storage disease and G6PD deficiency; disorders of amino acid metabolism such as phenylketonuria, maple syrup urine disease, and glutaric acidemia type 1; urea cycle disorders or defects such as carbamoyl phosphate synthetase I deficiency; disorders of organic acid metabolism (organic acidurias) such as alkaptonuria and 2-hydroxyglutaric acidurias; disorders of fatty acid oxidation and mitochondrial metabolism, such as medium-chain acyl-coenzyme A dehydrogenase deficiency (MCADD); disorders of porphyrin metabolism, such as porphyrias including acute intermittent porphyria; disorders of purine or pyrimidine metabolism such as Lesch-Nyhan syndrome; disorders of steroid metabolism such as lipoid congenital adrenal hyperplasia or congenital adrenal hyperplasia; disorders of mitochondrial function such as Kearsn-Sayre syndrome; disorders of peroxisomal function such as Zellweger syndrome; lysosomal storage disorders such as Gaucher's disease, multiple sulfatase deficiency, and Niemann-Pick disease; iron metabolism disorders including hemochromatosis and iron deficiency anemia; copper metabolism disorders including Wilson's disease, Menkes disease, and occipital horn syndrome; and lipid metabolism and storage disorders such as Wolman disease, cholesteryl ester storage disease, Fabry disease, Farber's disease, gangliosidoses, Krabbe disease, metachromatic leukodystrophy, primary carnitine deficiency, carnitine-acylcarnitine translocase deficiency, and carnitine palmitoyltransferase I or II deficiency. A person of skill in the art will identify the appropriate inhibitor for such a condition to be tested.

Ultimately, cell-free protein synthesis biosensors which rapidly and economically quantify metabolites and disease biomarkers at the point-of-care have the potential to reduce medical costs, individualize treatment, and improve patient outcomes.

E. coli-based CFPS reactions retain amino acid metabolism, but the open reaction environment facilitates metabolic engineering using small-molecule inhibitors and amino acid composition. We apply this approach to create CFPS reactions wherein the protein expression yield is highly sensitive to the amount of Gln added through the sample of interest. Furthermore, we demonstrate that this sensitivity is exhibited in the presence of human serum. The assay's detection range is ideally suited to sensing physiologically relevant Gln concentrations and could provide a useful clinical tool to help individuals suffering from severe infection, anorexia nervosa, chronic kidney disease, diabetes, and cancer. The metabolic engineering approach used to create this sensor could also be applied to improve cell-free production, facilitate drug discovery, and create economic point-of-care sensors of other clinical biomarkers.

### EXAMPLES

Example 1: E. coli cell extract containing bacteriophage T7 RNA polymerase was prepared as previously described. Briefly, 5 mL overnight culture of BL21 Star™ (DE3) was added to 100 mL 2xYT. When OD600 reached ˜2.0, culture was transferred to 900 mL 2xYT. 1 L cultures were induced with 1 mM IPTG at OD600 0.4-0.6 and harvested at OD600 ˜2.5. All bacterial cultures were incubated at 37° C. and 280 RPM. Cells were homogenized with three passes at 21000 psi using the EmulsiflexB15 French press homogenizer (Avestin, Ottawa, Calif.) and centrifuged at 12000 RCF for 10 min. Clarified lysate was then incubated at 37° C. and 280 RPM for 30 min. Cell extract was dialyzed using 14 kDa MW cut-off cellulose membrane tubing (MilliporeSigma, Billerica, Mass.) for 4 hr at 4° C. immediately following incubation at 37° C.

Example 2: Cell-free protein synthesis reactions were formulated in 96-well plates with the following specifications: 25% (v/v) cell extract, 12 nM pY71-sfGFP³⁷, 10-20 mM magnesium glutamate (concentration adjusted for optimal protein yield), 1 mM 1,4-diaminobutane, 1.5 mM spermidine, 40 mM phosphoenolpyruvate, 10 mM ammonium glutamate, 175 mM potassium glutamate, 2.7 mM potassium oxalate, 0.33 mM nicotinamide adenine dinucleotide, 0.27 mM coenzyme A, 1.2 mM ATP, 0.86 mM CTP, 0.86 mM GTP, 0.86 mM UTP, 0.17 mM folinic acid, 6 mM aspartate, 6 mM asparagine, and 2 mM of the 16 remaining proteinogenic amino acids. Gln was omitted from Gln-sensing reactions. For reactions containing human serum (Gemini Bio Products, Sacramento, Calif.), murine RNAse inhibitor (New England BioLabs, Ipswich MA) was added to a final concentration of 0.8 U/μL CFPS. Reactions were incubated at 37° C. in a BioTek SynergyMX plate reader, shaken for 30 seconds, and analyzed for fluorescence every 4-6 minutes at 480/510 nm excitation/emission wavelengths. Results were obtained after 60 minutes. Assay signal strength was calculated as the ratio of GFP RFU at saturating Gln concentrations to the GFP RFU at 0 μM Gln.

Example 3: A CFPS reaction is initiated with NDPs instead of NTPs to serve as a rapid screening tool for inhibitors of nucleoside-diphosphate kinases, which may be important in cardiovascular disease and cancer metastasis. For example, this type of biosensor can be used to guage Nme1 activity, Nme2 activity, or both. Rescue of the transcription/translation system (that is, production of signal from the reporter) indicates NDP kinase activity, (particularly, Nme1/Nme2 activity) and an expected baseline activity. Failure to synthesize reporter and generate a signal indicates a possible deficiency in Nme1 and/or Nme2.

While a limited number of aspects have been described, those skilled in the art, having benefit of the above description, will appreciate that other aspects may be devised which do not depart from the scope of the present disclosure. In addition, it should be noted that the language used in the specification has been principally selected for readability and instructional purposes, and may not have been selected to delineate or circumscribe the subject matter. Accordingly, the present disclosure is intended to be illustrative, but not limiting, of the scope of its contents.

1. An in vitro system for detecting glutamine in a sample, the in vitro system comprising:
at least one of a transcription system and a translation system;
an inhibitor of glutamine biosynthesis; and
a reporter of transcription and/or translation in the system;
wherein the reporter provides a change in signal proportionate to rescue of transcription and/or translation by the sample relative to a level of transcription and/or translation in the system when the sample is absent.

2. The in vitro system of claim 1, wherein the inhibitor causes a metabolic deficiency in the in vitro system.

3. The in vitro system of claim 1, wherein the signal is proportionate to a rate or an extent of transcription or translation of the in vitro system.

4. (canceled)

5. The in vitro system of claim 1, wherein the transcription system or the translation system comprises at least one of a cell extract and a purified enzyme.

6. The in vitro system of claim 1, wherein the transcription system or the translation system comprises a dehydrated mixture.

7. The in vitro system of claim 5, wherein the cell extract comprises Escherichia coli cell extract. Page 3 of 6

8. (canceled)

9. The in vitro system of claim 1, wherein the reporter comprises green fluorescent protein.

10. The in vitro system of claim 1, wherein the inhibitor inhibits a glutamine synthetase.

11. The in vitro system of claim 1, wherein the sample comprises human serum, and wherein the inhibitor degrades asparagine.

12. (canceled)

13. (canceled)

14. An in vitro system for measuring a biological activity in a sample, the in vitro system comprising:
a coupled transcription-translation system;
an inhibitor of the coupled transcription-translation system; and
a reporter of the biological activity;
wherein the reporter is inactive when the sample is absent, and wherein the reporter provides a signal proportionate to rescue of the biological activity by the sample.

15. The in vitro system of claim 14, wherein the inhibitor causes a metabolic deficiency in the coupled transcription-translation system.

16. (canceled)

17. (canceled)

18. The in vitro system of claim 14, wherein the coupled transcription-translation system comprises at least one of a cell extract and a purified enzyme.

19. The in vitro system of claim 14, wherein the coupled transcription-translation system comprises a dehydrated mixture.

20. The in vitro system of claim 14, wherein the cell extract comprises Escherichia coli cell extract.

21. (canceled)

22. The in vitro system of claim 14, wherein the reporter comprises green fluorescent protein.

23. The in vitro system of claim 14, wherein the inhibitor inhibits a glutamine synthetase.

24. The in vitro system of claim 14, wherein the inhibitor degrades asparagine.

25. (canceled)

26. A method of using the in vitro system of claim 1 to detect glutamine, the method comprising:
combining the sample with the in vitro system, and
quantifying the signal produced after a predetermined time.

27. (canceled)

28. (canceled)

29. The method of claim 26, wherein the sample is human serum.

30. The method of claim 26, wherein the human serum is obtained from a patient suspected of having at least one of severe infection, anorexia nervosa, chronic kidney disease, and diabetes.

31. (canceled)
